(19) United States
(12) Plant Patent Application Publication
(10) Pub. No.: US 2020/0154622 P1
(43) Pub. Date: May 14, 2020

(54) STRAWBERRY PLANT NAMED 'DRISSTRAWSIXTYSIX'

(50) ;

(71) Applicant: Driscoll's, Inc., Watsonville, CA (US)

(72) Inventors: Philip J. Stewart, Watsonville, CA (US); Renae R. Robertson, Watsonville, CA (US); Kevin Coons, Watsonville, CA (US); Joanne F. Coss, Watsonville, CA (US); Amy Marie Edmondson, Watsonville, CA (US); Agustin Renteria, Watsonville, CA (US)

(73) Assignee: Driscoll's, Inc., Watsonville, CA (US)

(21) Appl. No.: 16/350,432

(22) Filed: Nov. 14, 2018

(51) Int. Cl. A01H 6/74

(52) U.S. Cl. PLT/208

(57) ABSTRACT

A new and distinct variety of strawberry plant named ‘DrisStrawSixtySix’, selected for its high yield, firm fruit, and compact plant type, is disclosed.

Latin name: Botanical classification: Fragaria x ananassa.

Varietal denomination: The varietal denomination of the claimed variety of strawberry plant is ‘DrisStrawSixtySix’.

### BACKGROUND OF THE INVENTION

Cultivated strawberry is a hybrid species of the genus Fragaria that is grown worldwide for its fruit. Modern strawberry was first bred in Brittany, France, in the 18^{th} century by crossing Fragaria virginiana with Fragaria chiloensis. Strawberry fruit is an aggregate accessory fruit, with the fleshy part of the fruit being derived from the receptacle that holds the ovaries.

Strawberry varieties vary widely in color, size, shape, flavor, season of ripening, degree of fertility, and susceptibility to disease. Certain varieties vary in foliage, and some vary in the relative development of their reproductive organs. Typically, strawberry flowers appear hermaphroditic in structure, but function as either male or female. Generally, commercial production of strawberry plants involves propagation from runners and distribution as either plugs or bare root plants. Cultivation is either perennial or annual plasticulture. During the off season, strawberries can also be produced in greenhouses.

Strawberry fruit is widely appreciated for its characteristic bright red color, aroma, juicy texture, and sweetness. Strawberry fruit is a popular fruit that is generally consumed either fresh or in prepared foods, such as preserves and baked goods.

Strawberry is an important and valuable fruit crop. Accordingly, there is a need for new varieties of strawberry plants. In particular, there is a need for improved varieties of strawberry plant that are stable, high yielding, and agronomically sound.

### SUMMARY OF THE INVENTION

In order to meet these needs, the present invention is directed to an improved variety of strawberry plant. In particular, the invention relates to a new and distinct variety of strawberry plant (Fragaria x ananassa), which has been denominated as ‘DrisStrawSixtySix’.

Strawberry plant variety ‘DrisStrawSixtySix’ originated from a cross between the proprietary female parent ‘DrisStrawFortyFour’ (U.S. Plant Pat. No. 26,801) and the proprietary male parent ‘62R 83’ (unpatented). Progeny plants from this cross, including ‘DrisStrawSixtySix’, were asexually propagated via stolons in Shasta County, Calif. in April of 2011. Strawberry plant variety ‘DrisStrawSixtySix’ was later specifically identified and selected in Monterey County, Calif. in May of 2012.

‘DrisStrawSixtySix’ was subsequently asexually propagated via stolons, and underwent further testing at a farm in Santa Barbara County, Calif. for six years (2012 to 2017). The present variety has been found to be stable and reproduce true to type through successive asexual propagations via stolons.

‘DrisStrawSixtySix’ exhibits the following distinguishing characteristics when grown under normal horticultural practices in Santa Barbara County, Calif.:

1. Spreading growth habit;

2. Inflorescence beneath foliage;

3. Calyx attachment inserted in fruit; and

4. Day neutral type of bearing.

‘DrisStrawSixtySix’ was selected for its high yield, firm fruit, and compact plant type.

### DESCRIPTION OF THE DRAWINGS

This new strawberry plant is illustrated by the accompanying photographs which show fruit of the plant, as well as the flowers and leaves. The colors shown are as true as can be reasonably obtained by conventional photographic procedures. The photographs are of plants that are seven months old.

FIG. 1 illustrates whole fruit of variety ‘DrisStrawSixtySix’.

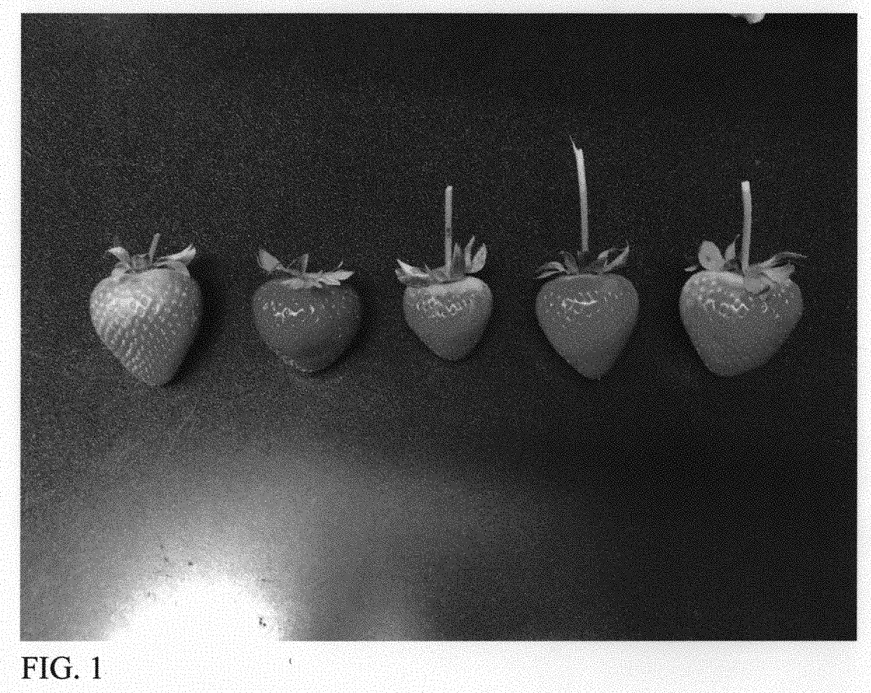

FIG. 2 illustrates longitudinal sections of fruit of variety ‘DrisStrawSixtySix’.

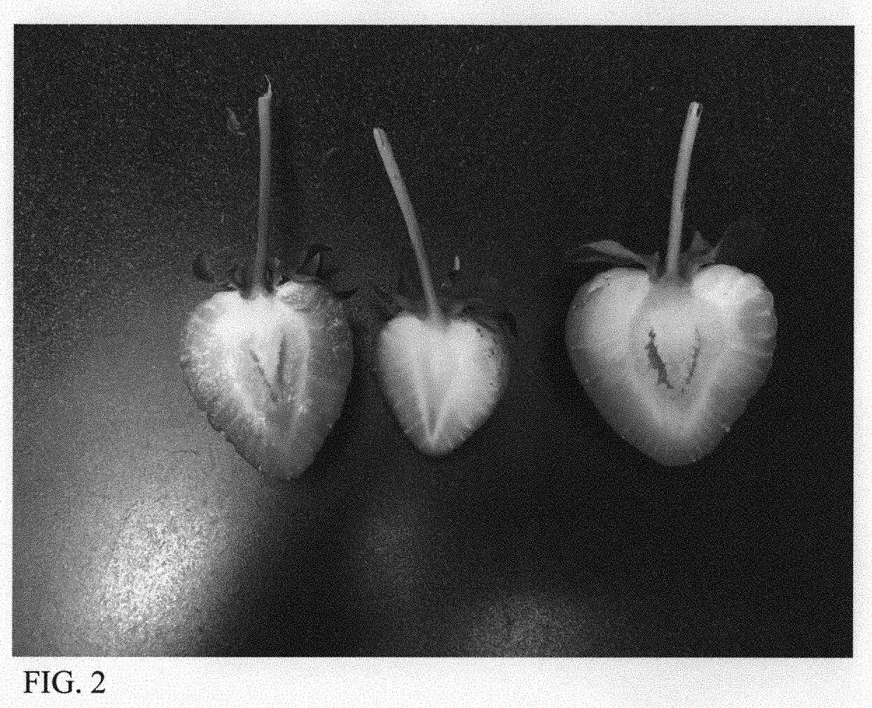

FIG. 3 illustrates the upper surfaces of flowers of variety ‘DrisStrawSixtySix’.

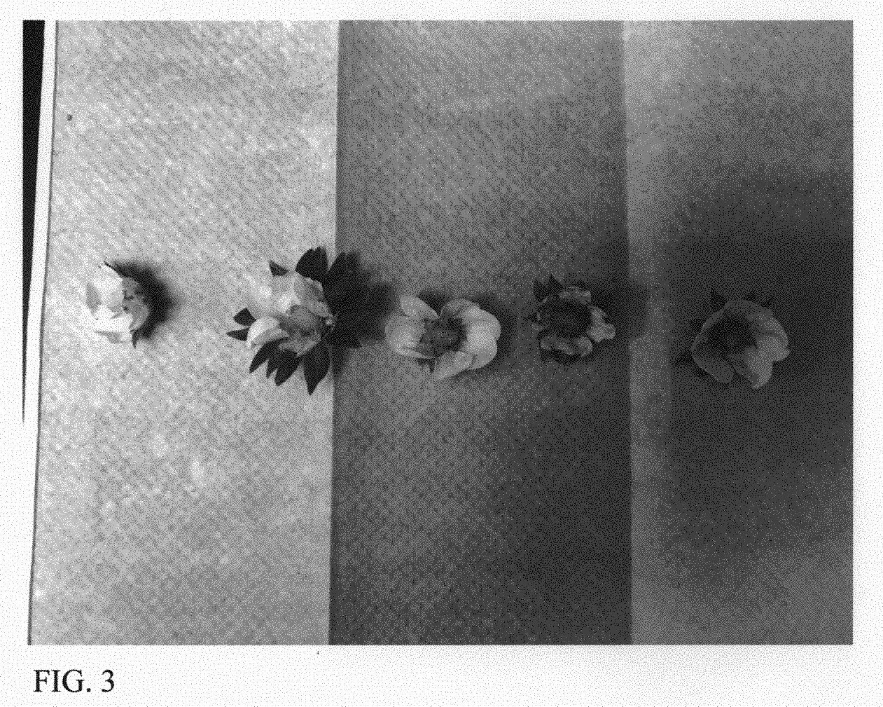

FIG. 4 illustrates the lower surfaces of flowers of variety ‘DrisStrawSixtySix’.

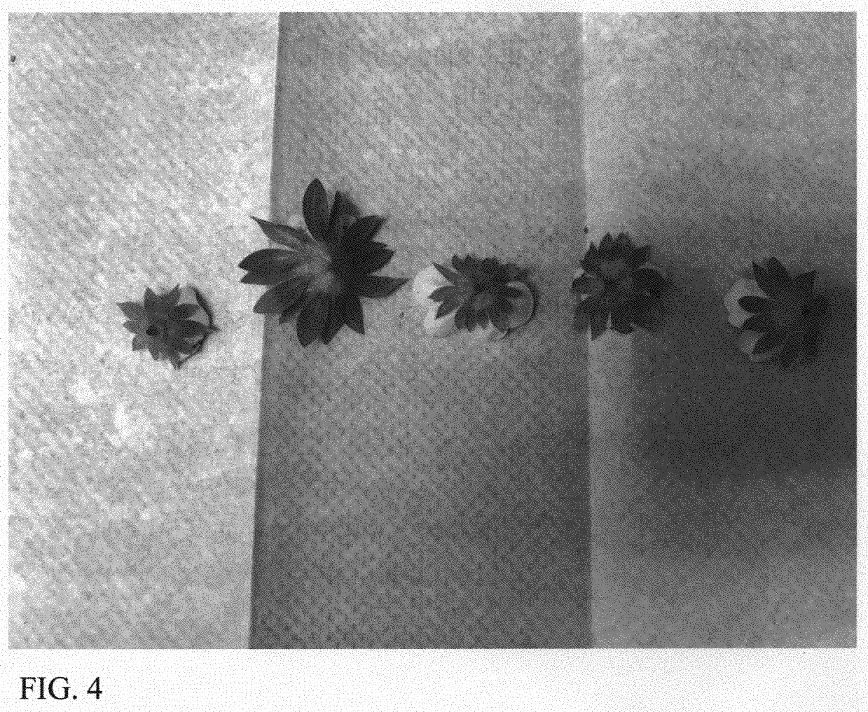

FIG. 5 illustrates leaves of variety ‘DrisStrawSixtySix’.

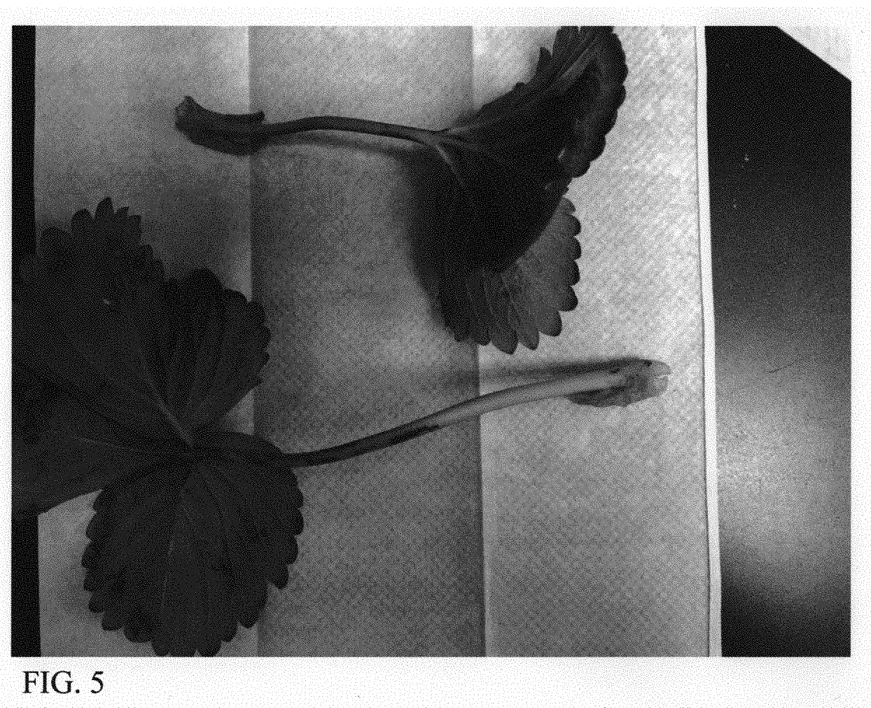

FIG. 6 illustrates fruit of variety ‘DrisStrawSixtySix’ at various stages of development.

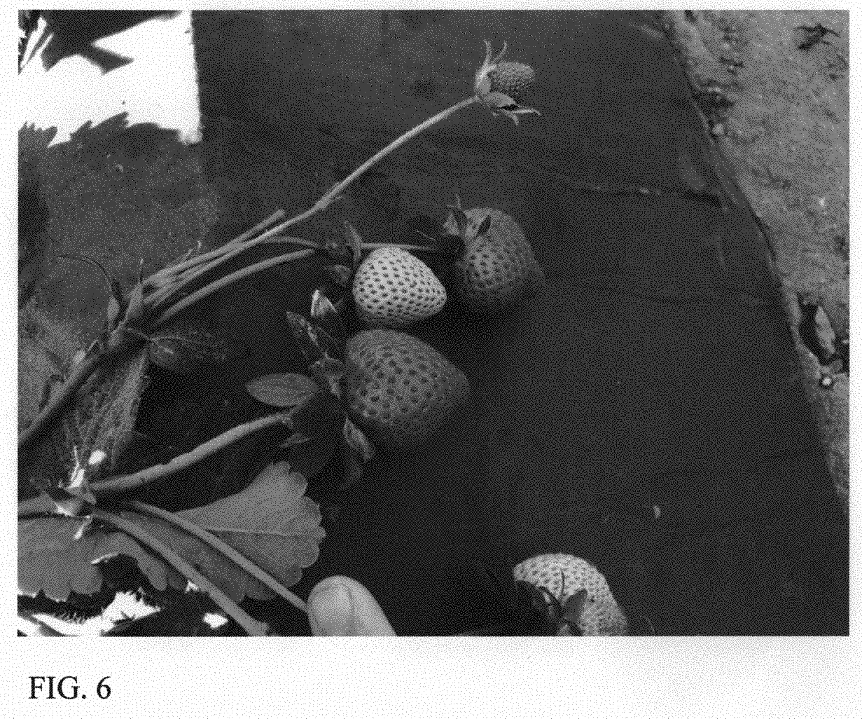

FIG. 7 illustrates plants of variety ‘DrisStrawSixtySix’.

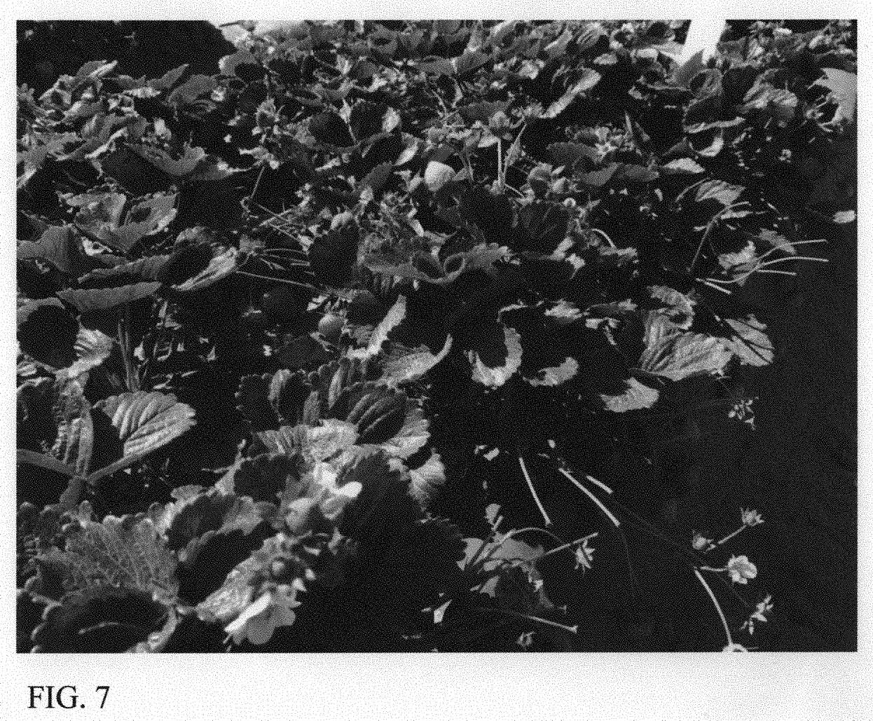

### DESCRIPTION OF THE NEW VARIETY

The following detailed descriptions set forth the distinctive characteristics of ‘DrisStrawSixtySix’. The data which define these characteristics is based on observations taken in Santa Barbara County, Calif. from 2012 to 2017. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawSixtySix’ has not been observed under all possible environmental conditions. The botanical description of ‘DrisStrawSixtySix’ was taken from plants that were seven months old. The indicated values represent averages calculated from measurements of several plants. Color references are primarily to The R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
   Species.—Fragaria x ananassa.
   Common name.—Strawberry.
   Denomination.—‘DrisStrawSixtySix’.
Parentage:
   Female parent.—The proprietary variety ‘DrisStrawFortyFour’ (U.S. Plant Pat. No. 26,801).
   Male parent.—The proprietary variety ‘62R 83’ (unpatented).
Plant:
   Height.—14.9 cm.
   Diameter.—28.3 cm.
   Number of crowns per plant.—3.
   Growth habit.—Spreading.
Stolon:
   Average number of daughter plants per square foot.—14.
   Diameter at bract.—2.60 mm.
   Anthocyanin coloration.—Present.
   Stolon color with anthocyanin color present.—RHS 162B (Moderate yellow).
   Length.—42 cm.
Leaf:
   Number of leaflets.—Three only.
   Color of upper surface.—RHS 136A (Dark green).
   Color of lower surface.—RHS 138B (Moderate yellow-green).
   Texture.—Coriaceous.
   Vein color.—RHS 138B (Moderate yellow-green).
   Venation Pattern.—Cross-venulate.
   Variegation.—Absent.
   Terminal leaflets.—Length: 5.9 cm. Width: 4.6 cm. Length/width ratio: 1.3. Number of teeth/terminal leaflet: 20. Shape of base: Rounded. Margin: Crenate. Shape in cross section: Concave.
   Petiole.—Length: 9.4 cm. Diameter: 2.10 mm. Attitude of hairs: Horizontal. Bract frequency (number present on each petiole): 0. Color: RHS 138A (Moderate yellowish green).
   Petiolule.—Length: 3.70 mm. Diameter: 2.00 mm.
   Stipule.—Length: 2.5 cm. Width: 7.20 mm. Anthocyanin color: RHS 149A (Brilliant yellow-green).
Inflorescence:
   Position in relation to foliage.—Beneath.
   Peduncle.—Length: 17 cm. Diameter: 4 mm. Color: RHS 137D (Moderate yellowish green).
   Pedicel.—Attitude of hairs: Upwards.
   Flower.—Shape: Cyanthiform. Flower diameter (petal tip to petal tip on non-flattened flower): 19.20 mm. Typical and observed number of flowers per plant: 5.60. Arrangement of petals: Overlapping. Stamen: Present.
   Petal.—Length: 8.30 mm. Width: 7.90 mm. Length/width ratio: 1.1. Typical and observed petal number: 5. Color of upper side: RHS 155B (Yellowish white). Color of lower side: RHS 155B (Yellowish white).
   Calyx.—Diameter (sepal tip to sepal tip, measured on back of flower): 41.40 mm.
   Sepal.—Length (sepal tip to point of attachment to receptacle): 12.50 mm. Width: 6.50 mm. Typical and observed sepal number: 11.
   Reproductive organs.—Pistil: Pistil length: 1.0 mm. Pistil width: 0.2 mm. Pistil shape: Simple. Pistil color: RHS 154B (Brilliant yellow). Stamen: Stamen length: 1.0 mm. Stamen width: 0.5 mm. Stamen shape: Basifixed. Stamen color: RHS 154C (Brilliant yellow-green). Pollen: Pollen diameter: 0.1 mm. Pollen shape: Ovulate. Pollen color: RHS 12A (Vivid yellow).
Fruit:
   Length.—38.80 mm.
   Width.—35.50 mm.
   Length/width ratio.—1.1.
   Fruit hollow length.—17.40 mm.
   Fruit hollow width.—10.10 mm.
   Fruit hollow length/width ratio.—1.7.
   Weight.—28.1 grams.
   Shape.—Conical.
   Color.—RHS 45A (Vivid red).
   Position of achenes.—Level with surface.
   Position of calyx attachment.—Inserted.
   Attitude of sepals.—Outwards.
   Color of flesh (excluding core).—RHS 44C (Vivid reddish orange).
   Color of core.—RHS 39B (Deep yellowish pink).
Production:
   Flowering interval.—February to September.
   Harvest interval.—March to September.
   Type of bearing.—Day neutral.
   Productivity.—84,189 lbs to 110,387 lbs of fruit per acre per season from seven-month-old plants when grown in Santa Barbara County, Calif.
Resistance to abiotic stress and diseases:
   Drought.—Moderately resistant.
   Heat.—Moderately resistant.
   Botrytis fruit rot (Botrytis cinerea).—Moderately susceptible.
   Powdery mildew (Podosphaera macularis).—Moderately susceptible.
   Anthracnose crown rot (Colletotrichum acutatum).—Susceptible.

### COMPARISON WITH PARENTAL AND COMMERCIAL VARIETIES

‘DrisStrawSixtySix’ differs from the proprietary female parent ‘DrisStrawFortyFour’ (U.S. Plant Pat. No. 26,801) in that ‘DrisStrawSixtySix’ is less vigorous but has increased fruit production and firmer fruit when compared with ‘DrisStrawFortyFour’.

‘DrisStrawSixtySix’ differs from the proprietary male parent ‘62R 83’ (unpatented) in that ‘DrisStrawSixtySix’ has increased fruit production and firmer fruit with fewer culls when compared with ‘62R 83’.

‘DrisStrawSixtySix’ differs from the commercial variety ‘DrisStrawFortyFour’ (U.S. Plant Pat. No. 26,801) in that plants of ‘DrisStrawSixtySix’ have a spreading growth habit, whereas plants of ‘DrisStrawFortyFour’ have a semi-upright growth habit. Further, plants of ‘DrisStrawSixtySix’ have inflorescence beneath foliage, whereas plants of ‘DrisStrawFortyFour’ have inflorescence above foliage. Moreover, plants of ‘DrisStrawSixtySix’ have a medium number of stolons, whereas plants of ‘DrisStrawFortyFour’ have many stolons. In addition, fruit of ‘DrisStrawSixtySix’ have a position of calyx attachment inserted in fruit, whereas fruit of ‘DrisStrawFortyFour’ have a position of calyx attachment level with fruit.

‘DrisStrawSixtySix’ differs from the commercial variety ‘DrisStrawNine’ (U.S. Plant Pat. No. 20,733) in that plants of ‘DrisStrawSixtySix’ have a spreading growth habit, whereas plants of ‘DrisStrawNine’ have an upright growth habit. Further, plants of ‘DrisStrawSixtySix’ have inflorescence beneath foliage, whereas plants of ‘DrisStrawNine’ have inflorescence above foliage. Moreover, fruit of ‘DrisStrawSixtySix’ have a position of calyx attachment inserted in fruit, whereas fruit of ‘DrisStrawNine’ calyx attachment level with fruit. In addition, plants of ‘DrisStrawSixtySix’ have a day neutral type of bearing, whereas plants of ‘DrisStrawNine’ have a fully remontant type of bearing.

We claim:

1. A new and distinct variety of strawberry plant named ‘DrisStrawSixtySix’ as shown and described herein.
